SEQUENCE LISTING (12 sequences)

SEQ ID NO: 1 - DNA, 21 bp, Homo sapiens
tgctttgaatgcgtcccagag

SEQ ID NO: 2 - DNA, 20 bp, Homo sapiens
aaagccgctcaactacatgg

SEQ ID NO: 3 - DNA, 20 bp, Homo sapiens
ggtttgaatgtcctcccgta

SEQ ID NO: 4 - DNA, 19 bp, Homo sapiens
gcccaggcttgcttaggta

SEQ ID NO: 5 - DNA, 20 bp, Homo sapiens
ggcagggtattccaacagac

SEQ ID NO: 6 - DNA, 20 bp, Homo sapiens
tcctgaggcttctgcattct

SEQ ID NO: 7 - DNA, 20 bp, Homo sapiens
cgaatattgcgcttttcaga

SEQ ID NO: 8 - DNA, 18 bp, Homo sapiens
agattccgtgggcgtagg

SEQ ID NO: 9 - DNA, 20 bp, Homo sapiens
cctcaccagcaacagaacaa

SEQ ID NO: 10 - DNA, 20 bp, Homo sapiens
tcagctccatcagctccttt

SEQ ID NO: 11 - DNA, 20 bp, Homo sapiens
ctcaaaggaaagcccatcag

SEQ ID NO: 12 - DNA, 20 bp, Homo sapiens
ttccatgtttagcgcttcct

(19) United States
(12) Patent Application Publication
(10) Pub. No.: US 2012/0021428 A1
(43) Pub. Date: Jan. 26, 2012

(54) METHOD FOR DIAGNOSIS OF CANCER AND MONITORING OF CANCER TREATMENTS

(76) Inventors: Marcus Otte, Graz (AU); Martina Schwarz, Graz (AU)

(21) Appl. No.: 13/262,363

(22) PCT Filed: Mar. 12, 2010
(86) PCT No.: PCT/EP2010/053152

§ 371 Date: Sep. 30, 2011

(30) Foreign Application Priority Data
Mar. 31, 2009 (EP) 09004687.1

(51) Int. Cl. C12Q 1/68

(52) U.S. Cl. 435/6.12; 435/6.14

(57) ABSTRACT

The present invention relates to a method for cancer diagnosis and for monitoring cancer treatments based on the analysis of the DNA fragmentation pattern of repetitive elements (preferably LINED or multi copy genes (preferably U1 RNA) identified in body fluid samples isolated from cancer patients.

### TECHNICAL FIELD

The present invention relates to a method for cancer diagnosis and for monitoring cancer treatments based on the analysis of circulating DNA, in particular on the analysis of a specific DNA fragmentation pattern of repetitive elements or multi copy genes identified in body fluid samples.

### BACKGROUND ART

Cell free genomic DNA, so called circulating DNA, which is present in serum, plasma and other body fluids (i.e. urine, ascites, etc.) at low concentrations (0.2 to 200 ng/ml) is highly degraded (Wang, et al., 2003, Cancer Res., 63 (14): 3966-8). Upon successful detection of microsatellite instabilities and tumor specific mutations (e.g. p 53, K-ras, EGFR) in circulating DNA derived from tumor patients (Sidransky, D. et al., 1991; Science, 252 (5006): 706-9; Kimura, H., et al., 2007, Br. J. Cancer, 97 (6): 778-84) it became obvious that genomic DNA originating from tumor tissue (circulating tumor DNA) is a component of circulating DNA in cancer patients (Sozzi, G. et al., 2001; Cancer Res., 61 (12): 4675-8; Diehl, F. et al., 2008; Nat. Med. 14 (9): 985-90, Epub 2007, July 31). The level of circulating DNA in tumor patients has been found to correlate with several biological processes, i.e. tumor size (Sunami, E. et al., 2008, Ann. N Y Acad Sci. 1137: 171-4) as well as medical interventions (Chan, K. C. A. et al., 2008, Clin. Cancer Res., 14 (13): 4141-5). In line, in WO 2006/128192 the use of free circulating DNA for diagnosis, prognosis, and treatment of cancer is claimed.

Recently detection and quantification of circulating DNA has been improved by the analysis of repetitive elements. The repetitive sequences account for at least 50% of the human genome. About 90% of those human repetitive sequences belong to transposable elements. Long Interspersed Elements (LINEs) are one of the superfamilies of those transposon-derived repeats and account for 20% of the human genome. Three LINE families, LINE1, LINE2, and LINE3, are found in the human genome. Among those families only LINE1 (L1) is capable of transposition and is most abundant (accounts for approximately 17% of human DNA). The size of the full-length L1 is about 6.1 kb. Over 500,000 sequences exist in the entire human genome (Cordaux, R., 2008, Proc Natl Sci USA, 105 (49): 19033-4, Epub 2008, December 4). The use of methylated or unmethylated L1 in diagnosing, predicting, and monitoring of cancer progression and treatment was disclosed in WO 2008/134596.

However, due to a lack of specificity, the level of circulating DNA is not a useful clinical marker for cancer diagnosis since too many confounding biological and physiological processes seem to affect the level of circulating DNA, thus hampering its clinical application in cancer diagnosis. New methods are needed which allow the specific detection of circulating tumor DNA within the circulating DNA sample. Recently, Ellinger, J., et al. disclosed in Journal of Urology, 2009, January, 181(1):363-71, Epub 2008, November 17 an increased level of cell free DNA in patients with testicular cancer by detecting 106 bp, 193 bp and 384 bp actin-beta DNA fragments.

The present invention provides a significant improvement in the sensitivity and specificity of the method for early diagnosis of cancer, diagnosis for recurrence of such cancers and monitoring upon corresponding treatments.

### SUMMARY OF THE INVENTION

The invention relates to a method of diagnosis of cancer, comprising the steps of:
(a) determining a DNA fragmentation pattern of repetitive elements or multi copy genes represented by 4 to 6 fragments of 80 to 500 bp in a body fluid sample isolated from a patient suspected to have cancer,
(b) comparing the DNA fragmentation pattern determined in step (a) with a DNA fragmentation pattern in a reference sample,
(c) determining a level of said DNA fragments which are differentially expressed in the sample isolated from the diagnosed patient compared to the reference sample,
   wherein the level of the DNA fragments, if substantially different from the level in the reference sample, indicates that the diagnosed patient is likely to be suffering from cancer.

Furthermore, the invention relates to a method of monitoring progress or recess of disease in a patient suffering from cancer and being under cancer treatment comprising the steps of:
(a) determining a DNA fragmentation pattern of repetitive elements or multi copy genes represented by 4 to 6 fragments of 80 to 500 bp in a body fluid sample isolated from the monitored patient at the end of treatment,
(b) comparing the DNA fragmentation pattern determined in step (a) with a DNA fragmentation pattern in a control sample isolated from the same individual before the treatment,
(c) determining a level of said DNA fragments which are differentially expressed in the sample isolated from the monitored treated patient compared to the control sample,
   wherein the level of the DNA fragments, if substantially different from the level of the DNA fragments in the control sample, indicates that the cancer is likely to be at a more advanced stage or less advanced stage in the monitored patient than in the control sample isolated from the same patient, or the monitored patient is likely to be less responsive or more responsive to the cancer treatment.

### BRIEF DESCRIPTION OF FIGURES

FIG. 1A: Determination of the mean DNA-fragmentation pattern of aftercare breast cancer patients (n=17, open circles, dashed line) and non diseased/healthy controls (n=10, closed circles) from serum derived DNA.

Serum of aftercare patients is taken before secondary intervention, DNA is isolated and pooled. All aftercare breast cancer patients develop a metastasis in brain or liver several months after blood sampling or are suspected to suffer from metastasis. DNA of healthy controls is isolated but not pooled. The DNA-fragmentation is analysed in both groups by LINE1 specific real-time PCR as described in Example 2. The DNA-fragmentation pattern of the patients differs significantly from the non diseased/healthy control group. In the amplicon range between ˜150 and ˜400 bp the relative amount of DNA is significantly increased as compared to non diseased/healthy controls and should be used for diagnosing recurrence. Therefore five or more DNA-amplicons may be measured in that range and used for the calculation of a DNA fragmentation index (DR). X-axis: DNA amplicon length (bp), Y-axis: normalized DNA amplicon level.

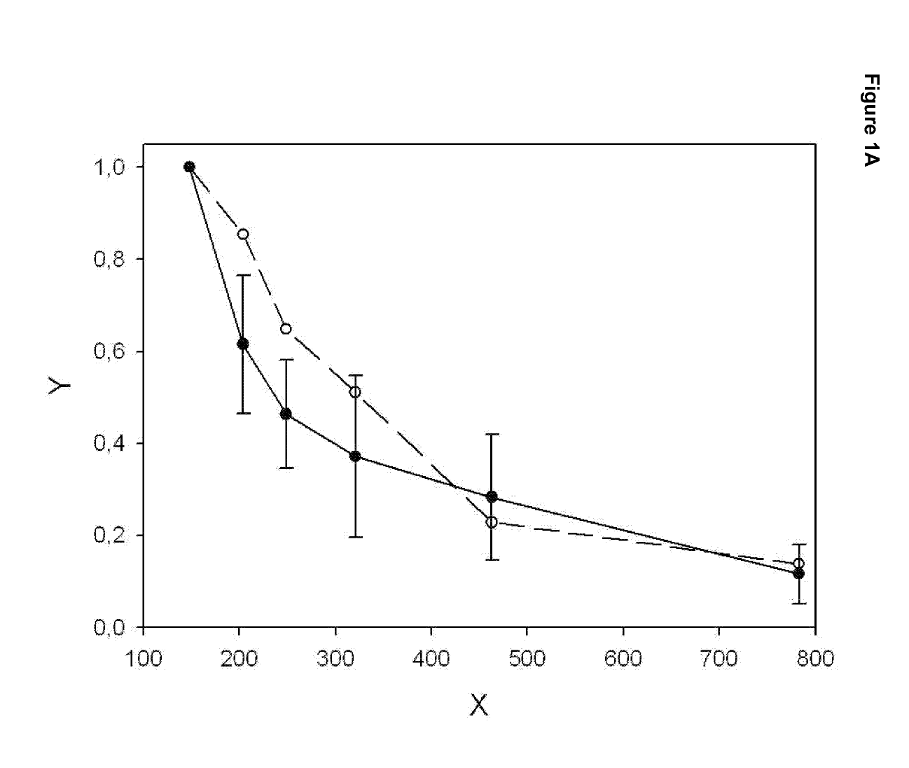

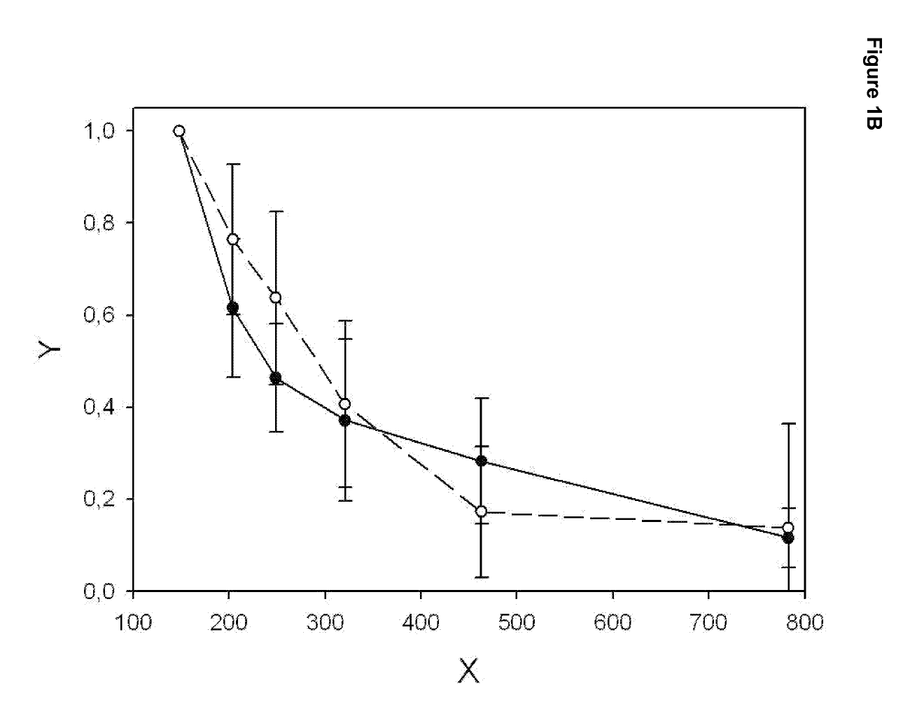

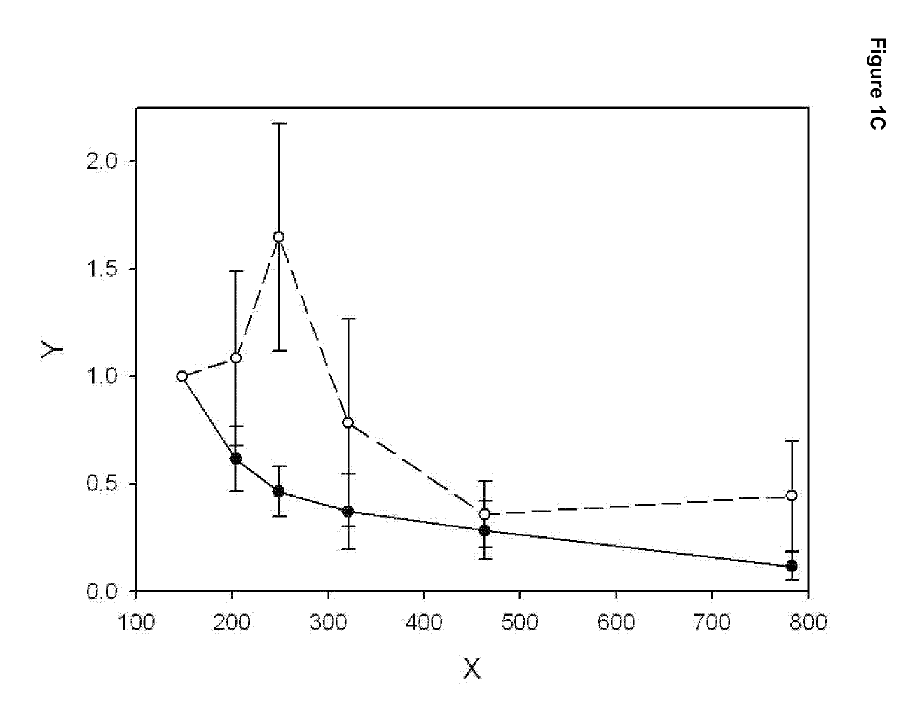

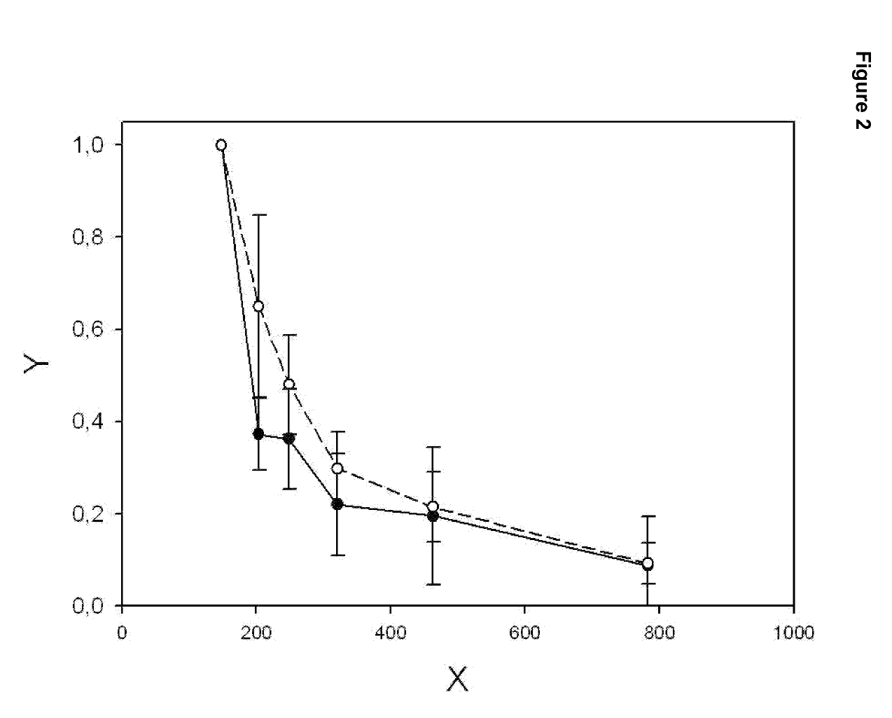

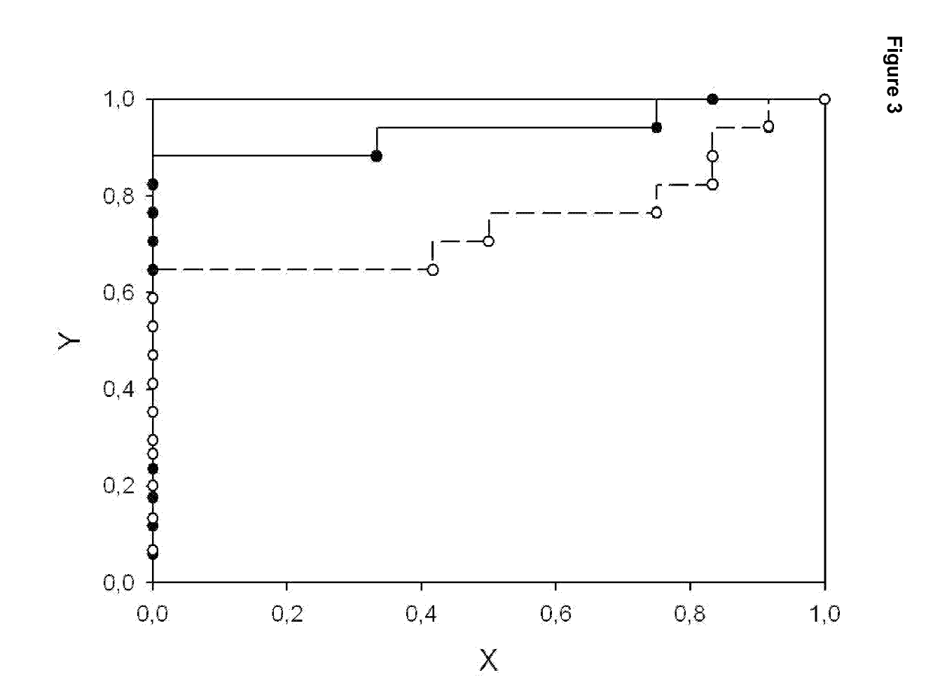

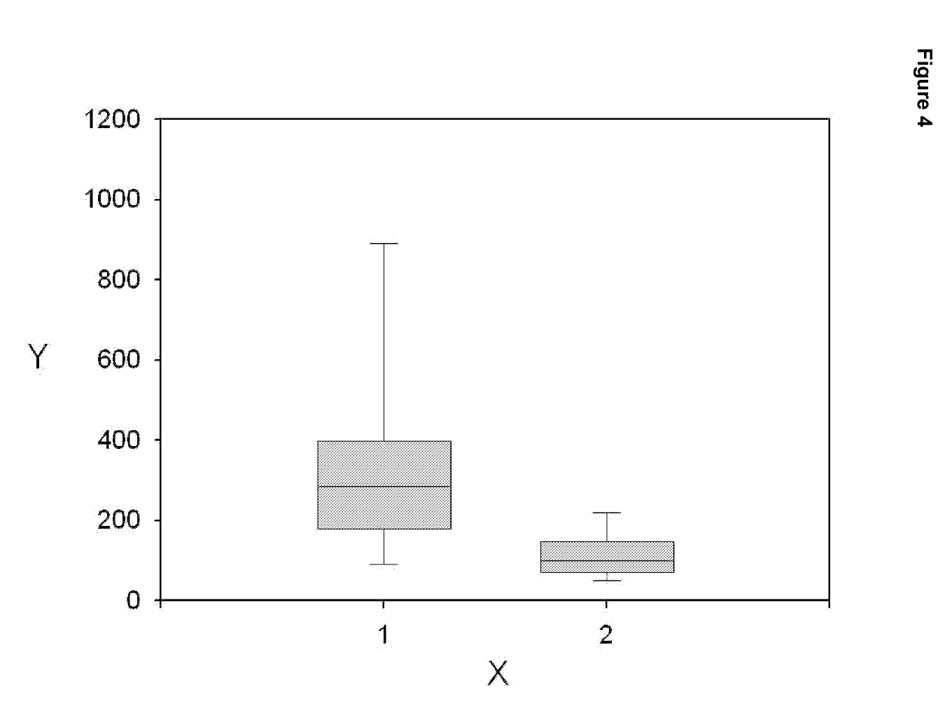

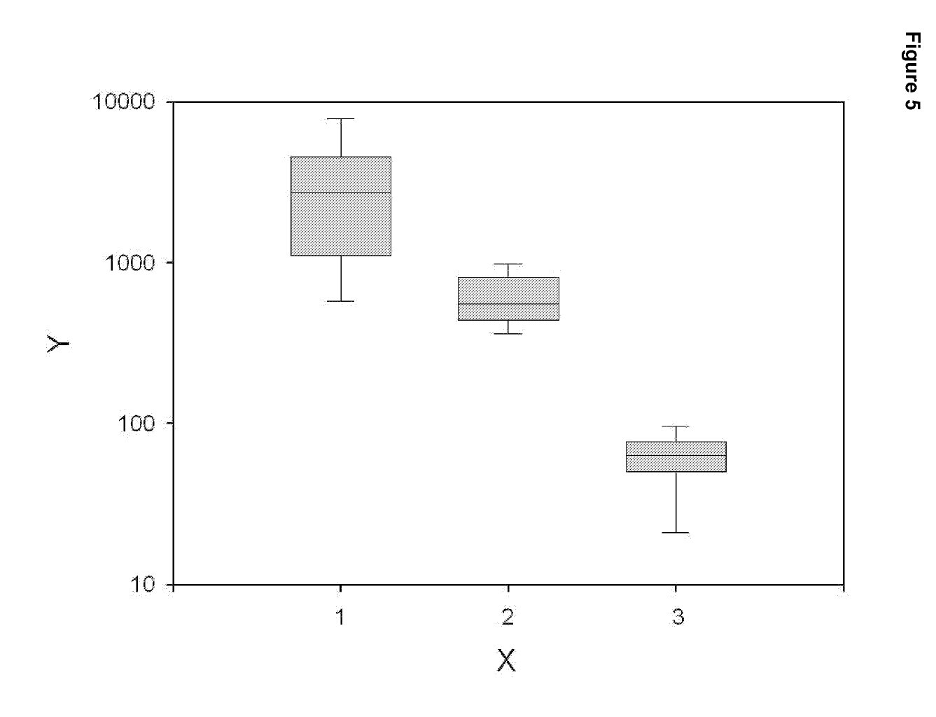

FIG. 1B: Determination of the mean DNA-fragmentation pattern of aftercare colon cancer patients (n=28, open circles, dashed line) and healthy controls (n=10, closed circles).

Serum of aftercare patients is taken before secondary intervention. All aftercare colon cancer patients develop a metastasis in the liver several months after blood sampling. DNA of both groups is isolated and analysed by LINE1 specific real-time PCR as described in Example 4. The DNA-fragmentation pattern of the patients differs significantly from the non diseased/healthy control group. In the amplicon range between ˜150 and ˜460 bp the relative amount of DNA is increased as compared to non diseased/healthy controls and should be used for diagnosing recurrence. Therefore five or more DNA-amplicons may be measured in that range and used for the calculation of a DNA fragmentation index (DFI). X-axis: DNA amplicon length (bp), Y-axis: normalized DNA amplicon level.

FIG. 1C: Determination of the mean DNA-fragmentation pattern of colon cancer patients with liver metastasis undergoing liver resection (n=33, open circles, dashed line) and healthy controls (n=10, closed circles).

Serum of cancer patients is taken at the day of liver resection. DNA of both groups is isolated and analysed by LINE1 specific real-time PCR as described in Example 4. The DNA-fragmentation pattern of the patients differs significantly from the non diseased/healthy control group. In the amplicon range between ˜150 and ˜780 bp the relative amount of DNA is increased as compared to non diseased/healthy controls especially at ˜250 bp and ≧600 bp. Therefore five or more DNA-amplicons may be measured in the range between 150-780 bp and used for the calculation of a DNA fragmentation index (DFI). However, the range between 150 and 460 bp is sufficient and same sensitivity/specificity is obtained as for the broader range. X-axis: DNA amplicon length (bp), Y-axis: normalized DNA amplicon level.

FIG. 2: Determination of the mean DNA-fragmentation pattern of de novo hepatocellular carcinoma patients (n=17, open circles, dashed line) and control patients suffering from liver cirrhosis (n=12, closed circles).

DNA of both groups is isolated and analysed by LINE1 specific real-time PCR as described in Example 3. The DNA-fragmentation pattern of the cancer patients differs significantly from the control group. In the amplicon range between ˜150 and ˜463 bp the relative amount of DNA is increased as compared to diseased control patients. Therefore five or more DNA-amplicons may be measured in the range between 150-460 bp and used for the calculation of a DNA fragmentation index (DFI). X-axis: DNA amplicon length (bp), Y-axis: normalized DNA amplicon level.

FIG. 3: Comparative ROC plot analysis. Diagnosis of HCC based on the DFI (closed circles) and alfa feto protein method (open circles, dashed line).

The DFI is estimated by the analysis of five LINE1 fragments (148, 204, 249, 321, and 463 bp) and computed by using equation [4.1] (see Example 3 for details). The area under the ROC plot for the DFI based (closed circles) diagnostic method is ˜0.89, and for the alfa feto protein (AFP) based (open circles, dashed line) method (i.e. ELISA) ˜0.75. These results indicate the superiority of the DNA-fragmentation pattern analysis for the diagnosis of HCC as compared to AFP. X-axis: False positive (1-specificity), Y-axis: True positive (sensitivity).

FIG. 4: Box plot diagram comparing the DNA-fragmentation index in HCC (n=17, left hand side) patients and at-risk patients with liver cirrhosis (n=12, right hand side).

The observed difference is statistically significant (p=0.004). The DFI is computed according to equation [4.1] after recording the relative level of five LINE1 fragments (148, 204, 249, 321 and 463 bp). 1=Patients suffering from hepatocellular carcinoma (HCC); 2=Patients suffering from liver cirrhosis, Y-axis: DFI (logarithmic scale).

FIG. 5: Box plot diagram comparing the DNA-fragmentation index in colon carcinoma patients with liver metastasis at the day of liver resection (1, left hand side), aftercare colon carcinoma patients up to 240 days before surgical or conventional treatment (2, middle) of liver metastasis and healthy controls (3, right hand side).

Five LINE1 fragments (148, 204, 248, 323 and 463 bp) are analyzed by real time PCR and the DFI is computed according to equation [4.1] as described in Example 4. The diagram exemplifies a clear discrimination of cancer patients from non diseased/healthy controls enabling a robust guess for the cut off value (in this example the DFI for diagnosing a relapse is >200). X-axis: 1=colon carcinoma patients with liver metastasis undergoing liver resection; 2=aftercare colon carcinoma patients prior liver metastasis treatment; 3=non diseased/healthy controls, Y-axis: DFI (logarithmic scale).

### DETAILED DESCRIPTION

It has now been unexpectedly found that a detailed analysis of the DNA fragmentation pattern (i.e. the relative abundance of DNA fragments of different sizes) of circulating DNA based on repetitive DNA elements (e.g. LINE1, SINE1, LTR) or multi copy genes (e.g. U1 RNA) by e.g. real-time PCR represents an improved method for cancer diagnosis and for monitoring cancer treatments. The DNA fragmentation pattern of healthy, at-risk patients and cancer patients differs significantly and therefore can be used for sensitive as well as specific cancer diagnosis and treatment monitoring (see Examples 1 to 4).

To gain clinical significant cancer specificity a DNA fragmentation pattern is represented by 4 to 6 fragments, more preferred 4, most preferred 5 fragments; in a length range of 50 to 2000 base pairs, more preferably 80 to 1200 base pairs, most preferably 80 to 500 base pairs. When five fragments in the most preferred range between 80 and 500 base pairs are used for determination of the DNA pattern, thresholds (cut offs) for the relative levels of tested DNA fragments are used for diagnosing cancer (a relative level of a DNA fragment is defined by the ratio of the amount of a tested DNA fragment and the amount of the shortest DNA fragment tested). The relative levels of the tested DNA fragments are the base for computing a DNA fragmentation index using an appropriate algorithm. In case of a DNA fragmentation pattern of 5 fragments the optimal size range is for a first fragment (A) 80-160 bp, for a second fragment (B′) 200-220 bp, for a third fragment (B) 240-260 bp, for a fourth (B″) fragment 300-380 bp, and for a fifth (C) fragment 400-500 bp. For example, when A=148 bp, B′=204 bp, B=249 bp, B″=321 and C=463 bp, thresholds for diagnosing cancer are A=1 (by definition), B′≧0.59, B≧0.46, B″≧0.29 and C≧0.13. The identified thresholds define the region in the DNA-fragmentation pattern plot indicative for the diagnosis of cancer.

The DNA fragments can be derived from the same gene/repetitive element or from different genes (e.g. LINE1, SINE, LINE2, U1 RNA etc.). For detection of LINE1 the DNA fragments are in a range of 50 and 2000 base pairs, preferably 80 to 1200 base pairs, most preferred 80 to 500 base pairs.

Overall, a sample analysis may be carried out in four subsequent steps. Step (1) isolation of circulating DNA, step (2) quantification of isolated DNA, step (3) quantification of DNA fragments by real time PCR, and finally step (4) data analysis and computation of the DNA fragmentation index (DFI), see a detailed protocol in Example 1.

The invention provides a method of determining whether a tested patient is suffering from cancer. In one such method, a body fluid sample is obtained from the patient to be diagnosed, and the level of DNA fragments of repetitive element(s) and/or multi copy genes, preferably LINE1, in the sample is determined. The level of the DNA fragments of a fragmentation pattern of LINE1 in the sample is compared with a control LINE1 DNA fragmentation pattern derived from a reference sample or a pool of reference samples, and the result of the comparison used to decide whether the subject is likely to be suffering from cancer. A reference sample is a sample obtained from a non diseased subject (healthy individual).

The invention also provides methods of monitoring cancer progression and treatment, as well as methods for predicting the outcome of cancer. These methods involve obtaining a body fluid sample from a patient suffering from cancer to be monitored, determining the level of fragments of repetitive element(s) and/or multi copy genes, preferably LINE1 DNA fragments, in the sample, and comparing it to a LINE1 DNA fragmentation pattern in a control sample from a patient or a pool of patients suffering from the corresponding cancer. A control patient may be a different patient suffering from the same type of cancer, or preferably the same patient at a different time point, e.g., at a different cancer stage, or before, during, or after a cancer treatment (e.g. surgery or chemotherapy).

When compared to the state of the art the method according to the invention surprisingly provides an improved, sustained and/or more effective method of diagnosing of cancer and/or monitoring of cancer treatment, in particular of primary liver cancers (e.g. hepatocellular carcinomas), secondary liver cancer derived from primary breast cancer, primary colorectal cancer, colon cancer, lung cancer, breast cancer, ovarian cancer, as well as a general method of monitoring relapse of cancer(s).

The general terms used hereinbefore and hereinafter preferably have within the context of this disclosure the following meanings, unless otherwise indicated.

The term “DNA fragmentation pattern” refers to the distribution of DNA fragments. The DNA fragments differ in length (i.e. number of base pairs) between 50 and 2000 base pairs. The DNA fragmentation pattern can be assessed by various quantitative and semi quantitative methods, especially by DNA amplification (i.e. PCR, LCR, IMDA) and DNA hybridization (i.e. array hybridization) but also by capillary electrophoresis. The minimal DNA fragmentation pattern used for diagnosis is represented by at least 4 DNA fragments but not more than 15 fragments differing in length; preferably between 4 and 6 fragments, more preferably 4, and most preferably 5 fragments. The minimal DNA fragmentation pattern for diagnosis is evaluated by recording the DNA fragments in size between 80 and 500 bp (see FIG. 1A, 1B, 1C and FIG. 2). The DNA fragmentation pattern is recorded in a disease positive and a clinical relevant disease negative control cohort. For optimal discrimination of the two cohorts at least 4 DNA fragments differing in length, preferably between 4 and 6 fragments, more preferred 4, and most preferred 5 fragments, are compared.

A DNA fragmentation pattern of cancer subjects is regarded as “substantially different”, if a difference of the DNA fragmentation pattern when compared to the clinical cohort (obtained by the analysis of the DNA fragmentation pattern in a reference sample or several reference samples) is statistically significant (i.e. p≧0.05). Statistical significance is analyzed by computing a mean DNA-fragmentation index and its standard deviation for each patient/healthy subject and comparing the DFI of two or more groups (i.e. cancer versus control) using standard statistical methods (i.e. Student's t-test etc.). The difference of the mean DNA-fragmentation index of two samples is interpreted as unequivocally different, if the difference is at least two times the mean standard deviation of these two samples.

The term “repetitive elements” (also known as “repetitive sequences”) defines sequences, wherein a particular DNA partial sequence is repeated at least four times. Repetitive elements account for at least 50% of the human genome. Two types of such elements are encompassed: (1) tandem repeats (i.e. microsatellites) and (2) transposable elements (i.e. short and long interspersed elements: SINESs and LINEs), the latter representing 90% of the overall human repetitive sequences. Three LINE families, LINE1, LINE2, and LINE3 account for 20% of the human genome. Among those families only LINE1 (L1) is capable of transposition and is most abundant (accounts for approximately 17% of human DNA).

The term “multicopy genes” relates to gene sequences with an approximate number of at least 8 copies, e.g. U1 RNA.

The term “body fluid” refers to any body fluid in which a cellular DNA or cells (e.g., cancer cells) may be present, including, without limitation, blood, serum, plasma, urea, bone marrow, cerebral spinal fluid, peritoneal/pleural fluid, pleural effusions, lymph fluid, spinal fluid, ascite, serous fluid, sputum, lacrimal fluid, stool, saliva and urine. Body fluid samples can be obtained from a patient using any of the methods known in the art.

The term “sample” refers to a biomaterial comprising the above defined “body fluid”. The sample can be isolated from a patient or another subject by means of methods including “invasive” or “non-invasive” methods. Invasive methods are generally known to the skilled artisan and comprise, for example, isolation of the sample by means of puncturing, surgical removal of the sample from the opened body or by means of endoscopic instruments. Minimally invasive and non-invasive methods are also known to the person skilled in the art. The term “minimally invasive” procedure refers to methods generally known for obtaining patient sample material that do preferably not require anesthesia, can be routinely accomplished in a physician office or clinic and are either not painful or only nominally painful. The most common example of a minimally invasive procedure is venupuncture. Preferably the “non-invasive” methods do not require penetrating or opening the body of a patient or subject through openings other than the body openings naturally present such as the mouth, ear, nose, rectum, urethra, and open wounds.

The term “reference sample” refers to a sample that serves as an appropriate control to evaluate the differential DNA fragmentation pattern according to the invention in a given sample isolated from a patient diagnosed for cancer; the choice of such appropriate reference sample is generally known to the person skilled in the art. Examples of reference samples include samples isolated from a non-diseased organ or tissue or cell(s) or body fluids of the same patient or from another subject, wherein the non-diseased organ or tissue or cell(s) or body fluid is selected from the group consisting of tissue or cells, blood, or the samples described above. For comparison of the level of the DNA fragmentation pattern in the sample isolated from a patient with disease, the reference sample may also include a sample isolated from a non-diseased organ or tissue or cell(s) of a different patient, wherein non-diseased tissue or cell(s) is selected from the sample group listed above. Moreover the reference may include samples from healthy donors, preferably matched to the age and sex of the patient.

The term “control sample” refers to a sample that serves as an appropriate positive control to evaluate the differential DNA fragmentation pattern according to the invention in a given sample isolated from a patient monitored for cancer; the choice of such appropriate control sample is generally known to the person skilled in the art. Examples of control samples include samples isolated from a diseased organ or tissue or cell(s) or body fluids of the same patient (at a different point in time) or from another subject, wherein the diseased organ or tissue or cell(s) or body fluid is selected from the group consisting of tissue or cells, blood, or the samples described above. For comparison of the level of the DNA fragmentation pattern in the sample isolated from a patient with disease, the control sample may also include a sample isolated from a diseased organ or tissue or cell(s) of a different patient, wherein diseased tissue or cell(s) is selected from the sample group listed above. Moreover the control may include samples from diseased donors, preferably matched to the age and sex of the patient.

As used herein, a “patient” refers to a human or animal, including all mammals such as primates (particularly higher primates), sheep, dog, rodents (e.g., mouse or rat), guinea pig, goat, pig, cat, rabbit, and cow, dead or alive. In a preferred embodiment, the subject is a human. In another embodiment, the subject is an experimental animal or animal suitable as a disease model. The patient is either suffering from cancer, preferably liver cancer (hepatocellular carcinoma), secondary liver cancer, breast cancer, colon cancer, lung cancer, prostate cancer, gastric cancer, ovarian cancer, including also relevant at risk groups for developing cancer (i.e. patients suffering from liver cirrhosis, patients with genetic predisposition for developing cancer, and aftercare patients), subject to analysis, preventive measures, therapy and/or diagnosis in the context of a disorder according to the invention.

As used herein, “cancer” refers to a disease or disorder characterized by uncontrolled division of cells and the ability of these cells to spread, either by direct growth into adjacent tissue through invasion, or by implantation into distant sites by metastasis. Exemplary cancers include, but are not limited to, carcinoma, adenoma, lymphoma, leukemia, sarcoma, mesothelioma, glioma, gerrainoma, choriocarcinoma, prostate cancer, lung cancer, breast cancer, colorectal cancer, gastrointestinal cancer, bladder cancer, pancreatic cancer, endometrial cancer, ovarian cancer, melanoma, brain cancer, testicular cancer, kidney cancer, skin cancer, thyroid cancer, head and neck cancer, liver cancer including primary (i.e. hepatocellular carcinoma) and secondary liver cancer, esophageal cancer, gastric cancer, intestinal cancer, colon cancer, rectal cancer, myeloma, neuroblastoma, and retinoblastoma. In addition the term cancer includes primary and secondary tumour sites. Preferably, the cancer is primary and secondary liver cancer, colorectal cancer, breast cancer, prostate cancer, lung cancer, ovarian cancer, gastric cancer, bladder cancer and kidney cancer.

The term “liver cancer” within the meaning of the invention includes carcinomas in the liver, preferably hepatocellular carcinoma (HCC), metastases in liver originated from any organ (e.g. colon, breast), cholangiocarcinoma, in which epithelial cell components of the tissue are transformed resulting in a malignant tumor identified according to the standard diagnostic procedures as generally known to a person skilled in the art. Preferably HCC further comprises subtypes of the mentioned disorders, preferably liver cancers characterized by intracellular proteinaceous inclusion bodies, HCCs characterized by hepatocyte steatosis, and fibrolamellar HCC. For example, precancerous lesions are preferably also included such as those characterized by increased hepatocyte cell size (the “large cell” change), and those characterized by decreased hepatocyte cell size (the “small cell” change) as well as macro regenerative (hyperplastic) nodules (Anthony, P. in MacSween et al, eds. Pathology of the Liver. 2001, Churchill Livingstone, Edinburgh). Within the meaning of the invention the term “disorder according to the invention” encompasses cancer as defined above, for example liver cancer, preferably HCC.

The term “treatment” within the meaning of the invention refers to a treatment that preferably cures the patient from a disorder according to the invention and/or that improves the pathological condition of the patient with respect to one or more symptoms associated with the disorder, preferably 3 symptoms, more preferably 5 symptoms, most preferably 10 symptoms associated with the disorder on a transient, short-term (in the order of hours to days), long-term (in the order of weeks, months or years) or permanent basis, wherein the improvement of the pathological condition may be constant, increasing, decreasing, continuously changing or oscillatory in magnitude as long as the overall effect is a significant improvement of the symptoms compared with a control patient.

The terms “more or less advanced stage” and “less or more responsive” is defined by clinical staging standards as provided by UICC/International Union Against Cancer: TNM staging (http://www.uicc.org/index.php?option=com_content&task=view&id=14275&Itemid=197) or AJCC/American Joint Committee on Cancer: Cancer Staging Manual/current 7^{th} edition issued on Jan. 1, 2010 (http://www.cancerstaging.org) reflecting a variety of clinical and pathological features of different types of cancer. Both staging systems are well known in the prior art and routinely implemented in clinical oncology. The significant responsiveness of the patient includes a reduction of primary and if present secondary (metastases) tumour's size(s) by 5%, more preferably 10 to 15%, or by change of the initial staging according to the above mentioned staging systems.

LINE1 DNA may exist as either “cellular” or “acellular” DNA in a subject. “Acellular DNA” refers to DNA that exists outside a cell in a body fluid of a subject or the isolated form of such DNA. “Cellular DNA” refers to DNA that exists within a cell or is isolated from a cell.

Methods for extracting acellular DNA from body fluid samples are well known in the art (Clausen, F. B. et al., 2007, Prenatal Diagn. 27 (1): 6-10; Ahmad, N. N. et al., 1995, J Med Genet, 32 (2): 129-30). Commonly, the acellular DNA in a body fluid sample is separated from cells by cell sedimentation, precipitated in alcohol, and dissolved in an aqueous solution. Methods for extracting cellular DNA from body fluid samples are also well known in the art (Sambrook, J. and Russel, D. W., 2001, Preparation and analysis of eukaryotic genomic DNA. In: Molecular Cloning—A Laboratory Manual, Volume 1, 3rd edition, pp 6.1-6.32, Cold Spring Harbor Laboratory Press, Cold Spring Harbor, N.Y.). Typically, cells are lysed with detergents. After cell lysis, proteins are removed from DNA using various proteases. DNA is then extracted with phenol, precipitated in alcohol, and dissolved in an aqueous solution. The presence of repetitive element(s) and/or multi copy gene(s), preferably LINE1 DNA, is/are then detected in the body fluid derived circulating DNA by using any of the methods well known in the art. Such methods include, but are not limited to DNA amplification methods (e.g. PCR, LCR and isothermal multi-displacement amplification), Southern blot, DNA sequencing, array hybridization, and capillary electrophoresis.

The invention relates to a method of diagnosis of cancer, comprising the steps of:
(a) determining a DNA fragmentation pattern of repetitive elements or multi copy genes represented by 4 to 6 fragments of 80 to 500 bp in a body fluid sample isolated from a patient suspected to have cancer,
(b) comparing the DNA fragmentation pattern determined in step (a) with a DNA fragmentation pattern in a reference sample,
(c) determining a level of said DNA fragments which are differentially expressed in the sample isolated from the diagnosed patient compared to the reference sample,
   wherein the level of the DNA fragments, if substantially different from the level in the reference sample, indicates that the diagnosed patient is likely to be suffering from cancer.

Furthermore, the invention relates to a method of monitoring progress or recess of disease in a patient suffering from cancer and being under cancer treatment comprising the steps of:
(a) determining a DNA fragmentation pattern of repetitive elements or multi copy genes represented by 4 to 6 fragments of 80 to 500 bp in a body fluid sample isolated from the monitored patient at the end of treatment,
(b) comparing the DNA fragmentation pattern determined in step (a) with a DNA fragmentation pattern in a control sample isolated from the same individual before the treatment,
(c) determining a level of said DNA fragments which are differentially expressed in the sample isolated from the monitored treated patient compared to the control sample,
   wherein the level of the DNA fragments, if substantially different from the level of the DNA fragments in the control sample, indicates that the cancer is likely to be at a more advanced stage or less advanced stage in the monitored patient than in the control sample isolated from the same patient, or the monitored patient is likely to be less responsive or more responsive to the cancer treatment.

In one preferred embodiment of the method according to the invention the DNA fragmentation pattern is represented by 5 fragments. In a further preferred embodiment of the invention the DNA fragmentation pattern represented by 5 fragments comprises a first fragment in a range between 80 and 160 bp, a second fragment in a range between 200 and 220 bp, a third fragment in a range between 240 to 260 bp, a fourth fragment in a range between 300 and 380 bp and a fifth fragment in a range between 400 and 500 bp.

One of the preferred embodiments of the repetitive element is LINE1, SINE1 or LTR and of the multi copy gene is U1 RNA. The most preferred embodiment of the repetitive element is LINE1.

In another preferred embodiment the body fluid is blood, serum, plasma, urine, bone marrow, peritoneal fluid, or cerebral spinal fluid. In a particularly preferred embodiment the body fluid is serum or plasma.

A preferred embodiment of cancer is liver, breast, colon, colorectal, lung, prostate, ovarian or gastric cancer. Further preferred embodiment of liver cancer is primary or secondary liver cancer. The most preferred embodiment of liver cancer is hepatocellular carcinoma (HCC).

It will be apparent to those skilled in the art that various modifications can be made to the compositions, methods and processes of this invention. Thus, it is intended that the present invention cover such modifications and variations, provided they come within the scope of the appended claims and their equivalents. All publications cited herein are incorporated in their entireties by reference.

The invention will be further illustrated below with the aid of the figures and examples, representing preferred embodiments and features of the invention without the invention being restricted hereto.

### EXAMPLES

### Example 1

### Methods of Sample Analysis and DNA Fragment Pattern Selection

Overall, sample analysis and fragment pattern selection is carried out in five subsequent steps. Step (1) isolation of circulating DNA, step (2) quantification of isolated DNA, step (3) quantification of DNA fragments by real time PCR, step (4) data evaluation and fragment pattern selection and finally step (5) computation of the DNA fragmentation index (DFI).

Step (1): Isolation of Circulating DNA

Circulating DNA is isolated from 1 to 2.5 ml frozen (−20° C.) or fresh serum samples using the Qiagen MinElute Virus Vacuum kit (Cat. No. 57714, Qiagen, Hilden, Germany) following the instructions of the manufacturer (Qiagen kit manual, 3^{rd} edition, March 2007, page 23-25). Frozen plasma samples (2-2.5 ml) are processed using the Qiagen MinElute Virus Spin kit (Cat. No. 57704, Qiagen, Hilden, Germany) following the instructions of the manufacturer (Qiagen kit manual, 3^{rd} edition, February 2007, page 19-22).

Step (2): Quantification of Isolated DNA

Isolated DNA, present in a total volume of up to 20 μl, is quantified using the PicoGreen assay (Cat. No. P11496, Invitrogen, Carlsbad, Calif., USA) following the instructions of the manufacturer (revised version 20 Dec. 2005/MP07581). The standard curve ranges from 1 to 100 ng human genomic DNA per ml (Applied Biosystems, Foster City, Calif., USA).

Step (3): Quantification of DNA Fragments by Real-Time PCR

For subsequent sample analysis by real-time PCR 0.02 ng to 0.1 ng of isolated circulating DNA is analysed per reaction. Four or more repetitive gene fragments of different length are analyzed in parallel by separate real-time PCR reactions. For each reaction the conditions are as follows: primer (see Table 1 & 2) concentration 50 nM each and final reaction volume of 25 μl using the 2×SYBR-Green master mix (Applied Biosystem, Foster City, Calif., USA). Samples are cycled in a two-step mode (see Table 3) for 40 cycles on a “7300 Real Time PCR System” (Applied Biosystem, Foster City, Calif., USA). Human genomic DNA (Applied Biosystems, Foster City, Calif., USA) is used as standard for relative DNA quantification of each repetitive gene fragments analyzed.

Step (4): Data Evaluation and Fragment Pattern Selection

For each individual patient the level of each tested gene fragment is normalized to the shortest gene fragment tested. To compare different patients or risk groups the mean level of the normalized gene fragment and its standard deviation is estimated for each amplicon size and each group. To obtain the DNA fragmentation pattern the mean normalized DNA amplicon level is plotted versus the amplicon size (see FIGS. 1A, 1B, 1C and 2). For each indication of interest the DNA fragmentation pattern is compared to a clinical relevant control cohort (see FIGS. 1A, 1B, 1C and 2). The clinical relevant control cohort presents the clinical subgroups which have to be discriminated by the diagnostic assay (e.g. liver cirrhosis vs. HCC, colon cancer/colon cancer recurrence vs. healthy). The DNA-fragmentation pattern of the disease groups shows a significant deviation form the clinical relevant control group in hepatocellular carcinoma, metastatic breast cancer and metastatic colon carcinoma. However, depending on the indication and the clinically relevant control groups, different characteristics of the DNA-fragmentation pattern curve (i.e. differences and changes of the slope of the curve, area under the curve) have to be selected for an optimal differentiation

As shown in Example 3 and FIG. 2, the slope of the DNA fragmentation pattern curve of HCC patients between amplicon size 149 and 463 bp differs significantly from the pattern of liver cirrhosis patients. Therefore, the region between amplicon size 149 and 463 bp is chosen for selection of a DNA-fragment pattern to diagnose HCC.

A similar region is selected for the diagnosis of breast cancer and breast cancer recurrence (see Example 2, FIG. 1A), as well as colon carcinoma and colon carcinoma recurrence (see Example 4, FIGS. 1B & 1C). The comparison of diseased patient and control donors indicates significant differences in the DNA fragmentation pattern. Therefore optimal diagnosis (high sensitivity and specificity) is obtained by the combination of the observed differences rather than using a single discriminator. This advantage is of importance when early or minimal residual disease status has to be diagnosed rather than highly advanced disease.

Step (5): Computation of the DNA Fragmentation Index (DFI)

For optimal discrimination of patients and controls the area under the DNA fragmentation pattern curve may be computed or empiric algorithms may be used to characterize the DNA fragmentation by a single FIGURE. Such a DNA fragmentation index (DFI) is further used to compare diseased and control patients and to define a threshold level (cut off level) for diagnosing cancer, to monitor treatment response as well as disease progression.

In an empiric algorithm the area under the curve as well as ratios and differences of normalized levels of gene fragments/amplicons are used in order to compute a DNA fragmentation index (DFI). The DNA fragments used for the calculation of the DFI are selected from the DNA fragmentation pattern of comparing clinical relevant groups (i.e. diseased versus control). In the example for diagnosing HCC the fragments are selected in the range between 148 and 463 bp (see Example 3). For three fragments the DFI is computed according to equation [1].

DFI₃=F(B)ₙₒᵣₘ/F(A)ₙₒᵣₘ×F(B)ₙₒᵣₘ/F(C)ₙₒᵣₘ×(F(B)ₙₒᵣₘ−F(C)ₙₒᵣₘ) [1]

For four fragments equation [2] is used and for more fragments the general equation [3] is used.

$$\begin{matrix}D F {I}_{4} = {F ( B )}_{norm} / {F ( A )}_{norm} \times {F ( D )}_{norm} / {F ( A )}_{norm} \times {F ( B )}_{norm} / {F ( C )}_{norm} \times {F ( D )}_{norm} / {F ( C )}_{norm} \times ( {F ( B )}_{norm} - {F ( C )}_{norm} ) \times ( {F ( D )}_{norm} - {F ( C )}_{norm} ) & [ 2 \} \\ D F {I}_{n} = {\prod }_{m = 1}^{n} ( {F ( {X}_{m + 1} )}_{norm} / {F ( {X}_{1} )}_{norm} ) \times {\prod }_{m = 1}^{n} ( {F ( {X}_{m + 1} )}_{norm} / {F ( {X}_{n} )}_{norm} ) \times {\prod }_{m = 1}^{n} ( {F ( {X}_{m + 1} )}_{norm} - {F ( {X}_{n} )}_{norm} ) & [ 3 ]\end{matrix}$$

### Legend:

DFIₙ: DNA fragmentation index calculated using n fragments A, B, C, D indicate four different amplicon sizes with the order A<B<C<D . . . .
F(A), F(B), F(C), F(D), F(X): relative concentration of fragment A, B, C, D, and X, respectively, per ml body fluid
F(A)ₙₒᵣₘ: F(A) normalized to F(A), i.e. 1.
F(B)ₙₒᵣₘ, F(C)ₙₒᵣₘ, F(D)ₙₒᵣₘ: F(B), F(C), and F(D), respectively, normalized to F(A) X₁, X₂ . . . Xₙ indicate different amplicon sizes with the order X₁<X₂< . . . <Xₙ
F(X)ₙₒᵣₘ: F(X) normalized to F(X₁); X₁ being the shortest DNA fragment)

Alternative more specific calculations, especially for colon and breast cancer but also for other cancers like HCC, are based on the analysis of at least five DNA fragments. Here the area under the DNA fragmentation plot (e.g. estimated by graphical integration) in the amplicon range between 130 and 360 bp (“Area 130-360”) or between 130 bp and 430 bp (“Area 130-430”); and multiplied by the amplicon ratio of fragments between 230 and 270 (F(B)ₙₒᵣₘ) and 430 and 470 (F(C)ₙₒᵣₘ), respectively (see equation 4). For the estimation of the area under the curve at least four different amplicon sizes should be tested (A, B, B′ and B″).

For the tested fragment the order of size is as follows: A<B′<B<B″<C.
The optimal size range for each fragment is as follows:

A=80-160 bp

B′=200-220 bp

B=240-260 bp

B″=300-380 bp

C=400-500 bp

Area=Area(130-360) or Area(130-430)

DFIₐᵣₑₐ=Area×F(B)ₙₒᵣₘ/F(C)ₙₒᵣₘ [4]

In summary fragments for optimal DFI calculation are selected after recording of the DNA-fragmentation pattern by comparing a disease positive and a clinical relevant disease negative control cohort (see plot normalized DNA amplicon level versus. DNA amplicon size).

It is found, that the DNA fragmentation pattern of cancer patients differs significantly from clinical relevant control cohorts (see FIGS. 1A, 1B, 10 and FIG. 2). The minimal DNA fragmentation pattern for cancer patient diagnosis records three DNA fragments:

1) F(A): a short DNA fragment between 80 and 150 bp in length

2) F(B): a DNA fragment between 200 and 270 bp in length

3) F(C): a DNA fragment between 360 and 500 bp in length

For a more specific and sensitive method of diagnosis and/or monitoring of cancer more than three fragments are used, namely additional fragments between 200 and 400 by are selected (see Example 3 and 4). It is confirmed that methods using four and more (preferably five) fragments are superior to methods using only two or three fragments (see FIG. 5).

In general the DNA fragmentation pattern and subsequently the DFI can be determined also by using SINE, U1 RNA, beta-actin or other repetitive elements or multi copy genes. To do so specific primers are designed, covering the same range of amplicon sizes. However, minor deviations in amplicon sizes (e.g. ±15 bp) due to primer design are negligible.

TABLE 1
Primer sequences for LINE1. Start/end indicate the position of
the first and last base of the primer annealing site with LINE1.

| | | | | primer | |
|---|---|---|---|---|---|
| | | | | length | |
| primer ID | primer sequence 5′->3′ | start | end | (bp) | direction |
| OrBi-178 | TGCTTTGAATGCGTCCCAGAG | 2848 | 2868 | 21 | r |
| OrBi-177 | AAAGCCGCTCAACTACATGG | 2720 | 2739 | 20 | f |
| OrBi-219 | GGTTTGAATGTCCTCCCGTA | 1073 | 1092 | 20 | r |
| OrBi-223 | GCCCAGGCTTGCTTAGGTA | 452 | 470 | 19 | f |
| OrBi-224 | GGCAGGGTATTCCAACAGAC | 772 | 791 | 20 | f |
| OrBi-226 | TCCTGAGGCTTCTGCATTCT | 1215 | 1234 | 20 | r |
| OrBi-250 | CGAATATTGCGCTTTTCAGA | 284 | 303 | 20 | f |
| OrBi-253 | AGATTCCGTGGGCGTAGG | 359 | 376 | 18 | r |
| OrBi-259 | CCTCACCAGCAACAGAACAA | 986 | 1005 | 20 | f |
| OrBi-261 | TCAGCTCCATCAGCTCCTTT | 1170 | 1189 | 20 | r |
| OrBi-263 | CTCAAAGGAAAGCCCATCAG | 1633 | 1652 | 20 | f |
| OrBi-264 | TTCCATGTTTAGCGCTTCCT | 1862 | 1881 | 20 | r |

R: reverse primer;
F: forward primer.
For primer combinations see Table 2.

TABLE 2
LINE1 fragments analyzed by real-time PCR.

| | | amplicon |
|---|---|---|
| LINE1 fragment ID | Primer IDs | size (bp) |
| LINE1-A | OrBi-250/OrBi-253 | 93 |
| LINE1-B | OrBi-177/OrBi-178 | 148 |
| LINE1-C | OrBi-259/OrBi-261 | 204 |
| LINE1-D | OrBi-263/OrBi-264 | 249 |
| LINE1-E | OrBi-224/OrBi-219 | 321 |
| LINE1-F | OrBi-224/OrBi-226 | 463 |
| LINE1-G | OrBi-223/OrBi-226 | 783 |

Primers designed for amplification of different amplicon sizes (bp). For primer sequences see Table 1.

TABLE 3
Cycling conditions.

| | Cycle | Temperature | Time |
|---|---|---|---|
| Process | number | (° C.) | (s) |
| Initial melting | 0 | 95 | 600 |
| Melting | 1-40 | 95 | 15 |
| Annealing/Extension | 1-40 | 72 | 90 |
| Melting curve analysis | n.a. | 65-95 | — |

### Example 2

### Relapse Diagnosis of Metastatic Breast Cancer by DNA-Fragmentation Analysis

The DNA fragmentation pattern of healthy donors (n=17) and a pool of patients (n=10) with metastatic breast cancer at various clinical stages is recorded (Example 1). LINE1 fragments with indicated by length are analyzed (see Table 2), normalized, and plotted (see FIG. 1B). The DNA fragmentation pattern (see FIG. 1A) of pooled breast cancer sera indicates an increased level of DNA fragments between 200 and 400 bp as compared to healthy controls.

The DNA fragmentation index (DFI) is computed for each patient/pool according to equation [2] (using LINE1-B, LINE1-C, LINE1-E and LINE1-F, see Table 2) as well as equation [4] (using LINE1-B, LINE1-C, LINE1-D, LINE1-E and LINE1-F, see Table 2).

The mean DFI₄ (equation [2]) for healthy control is 0.02±0.01 compared to 0.64 for the breast cancer pool; thus indicating a potential cut off around 0.1.

Similar results are obtained using equation [4]. The mean DFIₐᵣₑₐ is computed for the area between 148 and 323 bp and is 146±77 and 300.5 for non diseased/healthy controls and the breast cancer pool, respectively. A potential cut off may be around 200. The computation of the mean DFIₐᵣₑₐ for the area between 148 and 463 bp results in 193±82 and 449 for non diseased/healthy controls and the breast cancer pool, respectively. This results demonstrates a significantly improved discrimination/diagnosis of cancer (e.g. metastatic breast cancer) when compared to methods known in the prior art.

Additional improvement may be generated by the inclusion of characteristic amplicon ratios. As observed in FIG. 1A the slope of the non diseased/healthy control curve between amplicon 148 and 463 bp decreases faster then for metastatic breast cancer. A modification of equation [4] may lead to equation [4.1]:

DFIₐᵣₑₐ=Area(148-463)×F(B)ₙₒᵣₘ/F(C)ₙₒᵣₘ×F(B′)ₙₒᵣₘ/F(A)ₙₒᵣₘ×F(B″)ₙₒᵣₘ/F(C)ₙₒᵣₘ [4.1]

A=LINE1-B

B=LINE1-D

B′=LINE1-C

B″=LINE1-E

C=LINE1-F

Furthermore, by using equation [4.1] the mean DFIₐᵣₑₐ is computed to be 63±22 and 855 for non diseased/healthy controls and the breast cancer pool, respectively. A cut off for the DFIₐᵣₑₐ around 150 for diagnosing (metastatic) breast cancer is suggested. The inclusion of curve characteristics for the diseased group stepwise improves the power of discrimination of the two cohorts. As compared to the reported two-fragment based analysis using the Alu-fragment ratio of 247 bp/115 bp (Umetani, N. et al., 2006, Clin Chem. 52(6): 1062-9) results in a significant difference (healthy control=0.46±0.12 vs. 0.65 for breast cancer pool) but lacks diagnostic usability.

The advantage of analysing five DNA fragments in the range of 149 bp to 463 bp clearly indicates the superiority over two, three and four fragments. Such five DNA fragment analysis represents a significant improvement in cancer diagnosis, especially in clinical situations of minimal residual disease, early diagnosis and early recurrence detection.

### Example 3

### Diagnosis of Hepatocellular Carcinoma by DNA-Fragmentation Analysis

The DNA fragmentation pattern of patients with liver cirrhosis (at-risk patients) and patients with HCC is recorded as indicated in Example 1). LINE1 fragments with indicated sizes are analyzed (see Table 2), normalized, and plotted (see FIG. 2). FIG. 2 shows a significantly different curve of the DNA fragmentation pattern of HCC patients as compared to at risk patients suffering from liver cirrhosis. Therefore, the diagnosis of HCC may be obtained by recording the DNA fragmentation pattern in the indicated range of 148 to 783 bp, but at least between 148 and 463 bp and comparison with the DNA-fragmentation pattern of patients with liver cirrhosis and/or chronic hepatitis C. The DNA-fragmentation pattern of HCC patients differs most significantly from the control group between 204 and 463 bp.

Computation of DFI is performed according to equation [1], [2] and [4.1]. In order to optimize the LINE1 pattern defining the difference between patient and control group with highest sensitivity and specificity; the computation of the DNA fragmentation index focuses on the region between 148 and 463 bp. The data are summarized in Table 4 and 5. The area under the curve of the ROC plot increases from 2 to 5 LINE1 fragments (see Table 4), in addition the 95% confidence interval between patient and control groups are better separated when 4 and 5 LINE1 fragments are implemented (see Table 5), therefore the cut off value becomes more clear cut in these diagnostic tests. For the 4 and 5 fragment based diagnostic assay potential cut offs may be defined between the two non-overlapping confidence intervals (i.e. 0.9 and 196.0 for the 4 and 5 fragment based test, respectively). This is not possible for the 2 and 3 fragment based test (see Table 5) since the confidence intervals are overlapping.

A ROC plot (Altman, D. G. and Bland, J. M., 1994, Diagnostic tests 3: receiver operating characteristic plots, BMJ 309, 188; Zweig, M. H. and Campbell, G., 1993, Clin Chem. 39(4): 561-77) is performed (see FIG. 3) in order to show the superiority of the DNA fragmentation analysis as compared to alfa feto protein (AFP) analysis, the molecular standard diagnosis of HCC. The area under the ROC plot for AFP is 0.75±0.09 which indicates that a DNA-fragmentation-pattern based diagnosis is superior, independently how many LINE1 fragments are used (see Table 4). Also by box plot analysis (FIG. 4) the differentiation of cancer (i.e. HCC) and at-risk patients (i.e. cirrhosis) by DNA fragmentation pattern analysis becomes obvious.

Overall, the DNA fragmentation of HCC patients differs significantly from the control cohort of patients suffering from liver cirrhosis (see FIG. 2). The superiority of the DNA fragmentation pattern analysis as diagnostic tool as compared to AFP analysis, and the improvement by multi-DNA fragment analysis is clearly recognizable. Furthermore, there are several possibilities to compute a DFI based on the DNA fragments analysis. An optimal algorithm may represent the differences (i.e. sign and magnitude) and the changes (i.e. infliction point) of the slope of the DNA fragmentation pattern by combining appropriate fragment ratios and differences of the relative fragment levels. The general equation [3] (equation [1] and [2] are derived from the general equation [3] for 2 and 3 DNA fragments, respectively) and the equations [4] and [4.1] are given as examples for such algorithms. To expand the use of the diagnostic test for diagnosing HCC in at risk patients suffering from chronic hepatitis C or B, the DNA fragmentation pattern in these groups is recorded and analysed as indicated in Example 1 and 2. In principal, the same LINE1 fragments as used for the diagnosing HCC in at risk patents suffering from liver cirrhosis may be used for diagnosing HCC in at risk patients suffering from chronic hepatitis C or B.

TABLE 4
Comparing 2, 3, 4 and 5 LINE1 fragment based HCC diagnosis.

| No. of | | | | | |
|---|---|---|---|---|---|
| LINE1 | LINE1 | | | | Area under |
| frag- | amplicon | Equation | DFI | DFI | curve of |
| ments | size (bp) | used | (mean ± SD) | (median) | ROC plot |
| 2 | 148, 249 | Ratio of | 0.48 ± 0.11 | 0.46 | 0.82 ± 0.09 |
| | | 249/148 | | | |
| | | (Umetani | | | |
| | | et al., | | | |
| | | 2006) | | | |
| 3 | 148, 249, | [1] | 0.36 ± 0.29 | 0.25 | 0.78 ± 0.09 |
| | 483 | | | | |
| 4 | 148, 204, | [2] | 0.59 ± 0.92 | 0.23 | 0.90 ± 0.06 |
| | 249, 463 | | | | |
| 5 | 148, 204, | [4.1] | 351 ± 255 | 285 | 0.89 ± 0.06 |
| | 249, 321, | | | | |
| | 463 | | | | |

Four and five fragment based diagnostic methods are superior to two and three fragment based methods as indicated by the increase of the area under the ROC plot.

TABLE 5
Comparing 2, 3, 4 and 5 LINE1 fragment based HCC diagnosis.

| | HCC | | Liver cirrhosis | |
|---|---|---|---|---|
| No. of | | 95% | | 95% |
| LINE1 | DFI | confidence | DFI | confidence |
| fragments | (mean ± SD) | interval | (mean ± SD) | interval |
| 2 | 0.48 ± 0.11 | 0.42-0.54 | 0.36 ± 0.11 | 0.29-0.43 |
| 3 | 0.36 ± 0.29 | 0.21-0.51 | 0.15 ± 0.11 | 0.08-0.22 |
| 4 | 0.59 ± 0.92 | 0.12-1.06 | 0.036 ± 0.031 | 0.02-0.06 |
| 5 | 351 ± 255 | 219.9-482.1 | 111.3 ± 55.5 | 48.1-174.5 |

Four and five fragment based diagnostic methods are superior to two and three fragment based approaches as indicated by the comparison of mean and 95% confidence intervals of the two patient groups. The observed difference is of advantage for the definition of the cut off value. The DFI is computed as indicated in Table 4.

### Example 4

### Relapse Diagnosis of Metastatic Colon Cancer by DNA-Fragmentation Analysis

The DNA fragmentation pattern of healthy donors (n=10), patients with metastatic colon cancer undergoing liver resection (n=33) as well as of aftercare patients with colon cancer (n=28) are recorded (as indicated in Example 1). LINE1 fragments with amplicon length between 148 and 783 bp (see Table 2) are quantified by real time PCR, normalized, plotted and finally a DNA fragmentation index (DFI) is computed.

The serum derived DNA fragmentation pattern of healthy donors is compared with the serum derived DNA fragmentation pattern of colon carcinoma patients at two different time points and different clinical stages:
(1) patients with liver metastasis undergoing liver resection-time point of blood drawing: liver surgery (named group 1)
(2) aftercare patients with colorectal carcinoma-time point of blood drawing before surgery or conservative treatment of the liver metastasis (named group 2)

In FIG. 10 the DNA fragmentation pattern of non diseased/healthy controls versus group 1 is shown. The fragmentation patterns differ significantly. A maximum at around 250 bp and an increase of DNA fragments longer than 600 bp are dominating the fragmentation pattern of the cancer patients (see FIG. 10). The DNA fragmentation pattern of aftercare patients (group 2) with significantly lower tumour burden is shaping the DNA fragmentation pattern curve in a way that the maximum at ˜250 bp is reduced to a weak shoulder (see FIG. 1B). Therefore a sensitive diagnosis of distant tumour recurrence based on DNA fragmentation has to take the DNA-fragmentation pattern between 148 and 463 bp into account in order to accumulate the differences between the two patterns.

The DNA fragmentation index (DFI) is computed for both colon cancer groups using equation [4.1]. Accordingly a DFI threshold for disease free subjects, based on the non diseased/healthy control group, is estimated to approximately 100. A DFI of 200 may already indicate a relapse. The box plot in FIG. 6 compares the DFI (equation [4.1]) of group 1 patients and group 2 patients and non diseased/healthy controls. Both patient groups can be optimally separated from the healthy control group. The observed differences are statistically significant. In both clinical situations the sensitivity and specificity of the method is 100% (ROC plot not shown). These results indicate that the developed method is appropriate to monitor cancer patients (i.e. colon carcinoma, breast, cancer, prostate cancer, lung cancer etc.) for relapse diagnosis in order to achieve early diagnosis. This method may also be used as a surrogate marker of tumour response to new treatments. As shown already in Example 3, the diagnostic method may also be used as a marker for diagnosis of a primary tumour.

The superiority of the multi-fragment analysis is indicated in Table 6. In Table 6 a two fragment based index (Umetani et al. 2006, J. Clin. Oncol. 24 (26): 4270-6) is compared with the five fragment based DFI according to equation [4.1]. The indices for group 2 and non diseased/healthy controls are computed and finally the area under the ROC plot is estimated (comparing diseased versus non diseased/healthy control). The high sensitivity and specificity also indicates that the five-fragment-based assay could identify patients even earlier then tested in this study (the blood samples of the patients of group 2 are obtained up to 240 days before liver resection or conventional treatment). As already shown in Example 3, the selection of strong cut off values is improved by the five-fragment-based assay as compared to two-, three- or four-fragment-based assay(s).

TABLE 6
Comparing the diagnostic power of a five-
vs. a two- DNA-fragment- based assay.

| | Number of | DNA | Equation | |
|---|---|---|---|---|
| | DNA | amplicon | for index | Area under |
| Patient group | fragments | sizes (bp) | calculation | ROC plot |
| Group 2 vs. | 2 | 148, 249 | Umetani et al. | 0.78 ± 0.07 |
| Healthy | | | 2006 | |
| Group 2 vs. | 5 | 148, 204, 249, | 4.1 | 1 |
| Healthy | | 321, 463 | | |

### Example 5

### Surrogating Therapy Response of Cancer Treatment by DNA Fragmentation Analysis

The serum derived DNA fragmentation pattern of a cancer patient before and at the end of an anti-cancer treatment is recorded in triplicates. Subsequently from samples at both time points the mean DNA fragmentation index and its standard deviation is calculated. The mean DNA-fragmentation indexes in samples of both time points are compared with each other. A difference of the mean DNA-fragmentation indexes greater than two times the mean standard deviation is interpreted as unequivocally different. If the DNA-fragmentation index has unequivocally increased after treatment the patient is classified as non-responder. Responders are characterized by an unequivocal decrease of the DNA-fragmentation index at the end of the treatment. A difference of the DNA-fragmentation index which is not unequivocally different indicates no change and can be interpreted as stable disease. A clinical validation of the use of the DNA-fragmentation index as a surrogate for treatment response can be obtained by the correlation of the DNA-fragmentation index derived classification with the clinical outcome and/or with tumour-imaging data.

1. A method of diagnosis of cancer, comprising the steps of
(a) determining a DNA fragmentation pattern of repetitive elements or multi copy genes represented by 4 to 6 fragments of 80 to 500 bp in a body fluid sample isolated from a patient suspected to have cancer,
(b) comparing the DNA fragmentation pattern determined in step (a) with a DNA fragmentation pattern in a reference sample isolated from a patient not suffering from cancer,
(c) determining a level of said DNA fragments which are differentially expressed in the sample isolated from the diagnosed patient compared to the reference sample,
wherein the level of the DNA fragments, if substantially different from the level in the reference sample, indicates that the diagnosed patient is likely to be suffering from cancer.

2. The method according to claim 1, wherein the DNA fragmentation pattern is represented by 5 fragments.

3. The method according to claim 2, wherein the first fragment is in a range between 80 and 160 bp, the second fragment is in a range between 200 and 220 bp, the third fragment is in a range between 240 and 260 bp, the fourth fragment is in a range between 300 and 380 bp and the fifth fragment is in a range between 400 to 500 bp.

4. The method according to claim 1, wherein the repetitive element is LINE1, SINE1 or LTR and the multi copy gene is U1 RNA.

5. The method according to claim 1, wherein the repetitive element is LINE1.

6. The method according to claim 1, wherein the body fluid is blood, serum, plasma, urine, bone marrow, peritoneal fluid, or cerebral spinal fluid.

7. The method according to claim 6, wherein the body fluid is serum or plasma.

8. The method according to claim 1, wherein cancer is liver, breast, colon, lung, prostate, ovarian or gastric cancer.

9. The method according to claim 8, wherein liver cancer is primary and secondary liver cancer.

10. The method according to claim 9, wherein primary liver cancer is hepatocellular carcinoma.

11. A method of monitoring progress or recess of disease in a patient suffering from cancer and being under cancer treatment comprising the steps of:
(a) determining a DNA fragmentation pattern of repetitive elements or multi copy genes represented by 4 to 6 fragments of 80 to 500 bp in a body fluid sample isolated from the monitored patient at the end of treatment,
(b) comparing the DNA fragmentation pattern determined in step (a) with a DNA fragmentation pattern in a control sample isolated from the same individual before the treatment,
(c) determining a level of said DNA fragments which are differentially expressed in the sample isolated from the monitored treated patient compared to the control sample,
wherein the level of the DNA fragments, if substantially different from the level of the DNA fragments in the control sample, indicates that the cancer is likely to be at a more advanced stage or less advanced stage in the monitored patient than in the control sample isolated from the same patient, or the monitored patient is likely to be less responsive or more responsive to the cancer treatment.

12. The method according to claim 11 wherein the DNA fragmentation pattern is represented by 5 fragments.

13. The method according to claim 12, wherein the first fragment is in a range between 80 and 160 bp, the second fragment is in a range between 200 and 220 bp, the third fragment is in a range between 240 and 260 bp, the fourth fragment is in a range between 300 and 380 bp and the fifth fragment is in a range between 400 and 500 bp.

14. The method according to claim 11, wherein the repetitive element is LINE1, SINE1 or LTR and the multi copy gene is U1 RNA.

15. The method according to claim 11, wherein the repetitive element is LINE1.

16. The method according to claim 11, wherein the body fluid is blood, serum, plasma, urine, bone marrow, peritoneal fluid, or cerebral spinal fluid.

17. The method according to claim 16, wherein the body fluid is serum or plasma.

18. The method according to claim 11, wherein cancer is liver, breast, colon, lung, prostate, ovarian or gastric cancer.

19. The method according to claim 18, wherein liver cancer is primary and secondary liver cancer.

20. The method according to claim 19, wherein primary liver cancer is hepatocellular carcinoma.
